(19) United States
(12) Patent Application Publication
(10) Pub. No.: US 2007/0265196 A1
(43) Pub. Date: Nov. 15, 2007

(54) Method of Modulating Pro-Inflammatory and Inflammatory Activity Mediated by C-Reactive Protein

(75) Inventors: Pu Xia, Magill (AU); Carol Wadham, Coromandel Valley (AU); Mathew Vadas, Sterling (AU)

Correspondence Address: SCULLY SCOTT MURPHY & PRESSER, PC, 400 GARDEN CITY PLAZA, SUITE 300, GARDEN CITY, NY, 11530, US

(73) Assignee: MEDVET SCIENCE PTY., LTD., Adelaide, South Au. (AU)

(21) Appl. No.: 10/587,101

(22) PCT Filed: Jan. 21, 2005
(86) PCT No.: PCT/AU05/00071

§ 371 Date: Oct. 11, 2006

(30) Foreign Application Priority Data
Jan. 21, 2004 (AU) 2004900266

(51) Int. Cl. A61K 31/711; A61K 31/20; A61K 31/56; A61P 1/00; A61P 9/10; C12N 5/00; A61P 19/02; A61K 38/16; A61K 31/66

(52) U.S. Cl. 514/7; 435/375; 514/12; 514/143; 514/169; 514/2; 514/44; 514/558; 514/560

(57) ABSTRACT

The present invention relates generally to a method of modulating the proinflammatory activity of C-reactive protein in endothelial cells. More particularly, the present invention relates to down-regulation of proinflammatory activity of C-reactive protein in vascular endothelial cells. Accordingly, the method of the present invention is useful, inter alia, in the treatment and/or prophylaxis of inflammatory conditions, particularly conditions characterised by proinflammatory activity of C-reactive protein in endothelial cells.

### FIELD OF THE INVENTION

The present invention relates generally to a method of modulating the proinflammatory activity of C-reactive protein in endothelial cells. More particularly, the present invention relates to down-regulation of proinflammatory activity of C-reactive protein in vascular endothelial cells. Accordingly, the method of the present invention is useful, inter alia, in the treatment and/or prophylaxis of inflammatory conditions, particularly conditions characterised by proinflammatory activity of C-reactive protein in endothelial cells.

### BACKGROUND OF THE INVENTION

Bibliographic details of the publications referred to by author in this specification are collected alphabetically at the end of the description.

The reference to any prior art document in this specification is not, and should not be taken as, an acknowledgment or any form of suggestion that the document forms part of the common general knowledge in Australia.

Mammals are required to defend themselves against a multitude of pathogens including viruses, bacteria, fungi and parasites, as well as non-pathogenic insults such as disease and toxic, or otherwise harmful, agents. In response, effector mechanisms have evolved which are capable of mounting a defence against such antigens. These mechanisms are mediated by soluble molecules and/or by cells.

In the context of these effector mechanisms, inflammation is a complex multifaceted response to disease or injury which is regulated by the release of a cascade of cytokines. These cytokines are classified in general terms as pro- or anti-inflammatory cytokines and the critical balance between release and activity of cytokines with opposing actions regulates the inflammatory response to prevent it from becoming either overt or understated. If the inflammatory response continues unchecked and is overt then the host may suffer associated tissue damage. Conversely, a poor or understated inflammatory response may mean uncontrolled infection resulting in chronic illness and host damage. Regulation of the inflammatory response is important at both the systemic level and the local level.

The discovery of the detailed processes of inflammation has revealed a close relationship between inflammation and the immune response. These are five basic indicators of inflammation, these being redness (rubor), swelling (tumour), heat (calor), pain (dolor) and deranged function (funtio laesa). These indicators occur due to extravasation of plasma and infiltration of leukocytes into the site of inflammation. Consistent with these indicators, the main characteristics of the inflammatory response are therefore:
(i) vasodilation—widening of the blood vessels to increase the blood flow to the infected area;
(ii) increased vascular permeability—this allows diffusible components to enter the site;
(iii) cellular infiltration—this being the directed movement of inflammatory cells through the walls of blood vessels into the site of injury;
(iv) changes in biosynthetic, metabolic and catabolic profiles of many organs; and
(v) activation of cells of the immune system as well as of complex enzymatic systems of blood plasma.

The degree to which these characteristics occur is generally proportional to the severity of disease, injury or the extent of infection.

Inflammatory cardiovascular disease is a growing problem. Atherosclerotic coronary heart disease, for example, is one of the major causes of death in the western world (World Health Statistics Annual). An earlier event in atherogenesis is the adhesion of monocytes to the endothelium via adhesion molecules such as VCAM-1, ICAM-1 and E-selectin, all of which are rapidly synthesised in response to cytokines. VCAM-1 is primarily involved in the adhesion of mononuclear leukocytes to the endothelium. It is rapidly induced by the inflammatory cytokines IL-1 and TNF-α, and its induction is sustained for 48 to 72 hours. ICAM-1 is expressed on many cell types and is involved in both monocyte and lymphocyte adhesion to activated endothelium. E-selectin is an endothelial specific adhesion molecule important in capturing leukocytes from the axial stream to roll along the endothelium (Abbassi et al., 1993).

There is considerable evidence for the involvement of adhesion molecules in the development of early atherosclerotic lesions and in mature atherosclerotic plaques (Van der Wal et al., 1992). Variable and low levels of E-selectin and VCAM-1 have been detected in the arterial endothelium over plaques (Van der Wal et al., 1992; Wood et al., 1993). VCAM-1 has also been observed in areas of neovascularization and in inflammatory infiltrates at the base of plaques, suggesting that intimal neovascularization may be an important site of inflammatory cell recruitment into advanced coronary lesions (O'Brien et al., 1993). ICAM-1 has been shown to be expressed on the endothelium overlaying atheromatous plaques (Johnson-Tidey et al., 1994).

Accordingly, in light of the wide-ranging impact of inflammatory responses, there is an ongoing need to elucidate the complex mechanisms which lead to its upregulation, such as the mechanisms leading to expression of adhesion molecules.

In work leading to the present invention, the proinflammatory effect of C-reactive protein was measured by the induction of the inflammatory adhesion molecules E-selectin, VCAM-1 and ICAM-1 in human umbilical vein endothelial cells (HUVEC). It has been shown that C-reactive protein significantly induced upregulation of adhesion molecules in both protein and mRNA levels. The C-reactive protein-induced expression of these inflammatory adhesion molecules was completely suppressed when the cells were preincubated with a physiological concentration (1 mg/ml apoA-I) of high density lipoproteins (HDL) derived from human plasma (native HDL) or reconstituted HDL (rHDL) at a very low concentration (0.01 mg/ml apoA-I). In particular, the C-reactive protein-induced upregulation of inflammatory adhesion molecules in HUVEC was completely prevented by HDL via their oxidized phospholipid components. Further, it has been surprisingly determined that the mechanism by which HDL inhibits cytokine-induced adhesion differs so significantly from that by which HDL inhibits C-reactive protein-induced adhesion molecule expression that the modulation of TNF mediated adhesion molecule expression utilising HDL does not impact on C-reactive protein mediated adhesion molecule expression.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Accordingly, one aspect of the present invention provides a method of modulation of the endothelial cell proinflammatory phenotype which method comprises administration of an effective amount of an agent for a time and under conditions sufficient to modulate the functional activity of a C-reactive protein wherein down-regulating the functional activity of said C-reactive protein down-regulates said inflammatory phenotype.

The present invention therefore more particularly provides a method of modulation of endothelial cell adhesion molecule expression, which method comprises administration of an effective amount of an agent for a time and under conditions sufficient to modulate the functional activity of a C-reactive protein wherein down-regulating the functional activity of said C-reactive protein down-regulates said adhesion molecule expression.

The present invention therefore more preferably provides a method of modulation of vascular endothelial cell adhesion molecule expression, which method comprises administration of an effective amount of an agent for a time and under conditions sufficient to modulate the functional activity of a C-reactive protein wherein down-regulating the functional activity of said C-reactive protein down-regulates said adhesion molecule expression.

In a preferred embodiment, the present invention provides a method of down-regulating vascular endothelial cell adhesion molecule expression, which method comprises administration of an effective amount of an agent for a time and under conditions sufficient to down-regulate the functional activity of C-reactive protein.

In a still more preferred embodiment there is provided a method of down-regulating vascular endothelial cell adhesion molecule expression, which method comprises administration of an effective amount of an agent selected from:
(i) HDL
(ii) reconstituted HDL
(iii) PLPC
(iv) unsaturated phospholipid
   or a derivative thereof for a time and under conditions sufficient to down-regulate the functional activity of a C-reactive protein.

Accordingly, in a related aspect the present invention is directed to modulating the endothelial cell proinflammatory phenotype in a mammal, said method comprising administering to said mammal an effective amount of an agent for a time and under conditions sufficient to modulate the functional activity of a C-reactive protein wherein down-regulating the functional activity of said C-reactive protein down-regulates said inflammatory phenotype.

More particularly, there is provided a method of modulating endothelial cell adhesion molecule expression in a mammal, said method comprising administering to said mammal an effective amount of an agent for a time and under conditions sufficient to modulate the functional activity of a C-reactive protein wherein down-regulating the functional activity of said C-reactive protein down-regulates said adhesion molecule expression.

Most preferably, there is provided a method of down-regulating vascular endothelial cell adhesion molecule expression, said method comprising administering an effective amount of an agent for a time and under conditions sufficient to down-regulate the functional activity of a C-reactive protein.

Accordingly, the present invention is directed to modulating an inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of endothelial cells.

More particularly, there is provided a method of modulating an inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the adhesion molecule expression of endothelial cells.

Most preferably, there is provided a method of down-regulating an inflammatory response, said method comprising administering an effective amount of an agent for a time and under conditions sufficient to down-regulate the functional activity of a C-reactive protein, wherein down-regulating said C-reactive protein functional activity down-regulates endothelial cell adhesion molecule expression.

In yet another aspect, the present invention contemplates a method of therapeutically and/or prophylactically treating a condition or a predisposition to the development of a condition, characterised by an aberrant inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of an endothelial cell.

More particularly, there is provided a method of therapeutically and/or prophylactically treating a condition or a predisposition to the development of a condition, characterised by an aberrant inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the adhesion molecule expression of an endothelial cell.

The present invention therefore preferably provides a method of therapeutically and/or prophylactically treating atherosclerosis, atherosclerotic cardiovascular disease, inflammatory cardiovascular disease, diabetic vascular complication or a chronic inflammatory disease in a mammal, said method comprising administering to said mammal an effective amount of an agent for a time and under conditions sufficient to down-regulate the functional activity of a C-reactive protein wherein down-regulating the functional activity of said C-reactive protein down-regulates the adhesion molecule expression of the vascular endothelial cells.

In another preferred embodiment, said condition is obesity, diabetes and/or age. Patients exhibiting these conditions correspond to patients exhibiting a predisposition to the development of a condition characterised by an inflammatory response. Without limiting the present invention to any one theory or mode of action, these are examples of conditions which decrease the quantity and/or quality of HDL. Accordingly, they are often associated with an increase in C-reactive protein concentrations which can ultimately disturb the anti- vs pro-inflammatory balance thereby contributing to the development of inflammatory cardiovascular disease, for example. Accordingly, the method of the present invention may be valuable as a prophylactic measure to be applied to patients exhibiting this type of predisposition.

In yet another preferred embodiment, the present invention contemplates a method of therapeutically and/or prophylactically treating a condition or a predisposition to the development of a condition, which condition is characterised by an inadequate inflammatory response in a mammal, said method comprising administering to said mammal an effective amount of an agent for a time and under conditions sufficient to up-regulate the functional activity of a C-reactive protein wherein up-regulating the functional activity of said C-reactive protein up-regulates the proinflammatory phenotype of an endothelial cell.

Another aspect of the present invention relates to the use of an agent capable of modulating the functional activity of a C-reactive protein in the manufacture of a medicament for the therapeutic and/or prophylactic treatment of a condition, or a predisposition to the development of a condition, characterised by an aberrant inflammatory response in a mammal wherein down-regulating the functional activity of said C-reactive protein down-regulates the proinflammatory phenotype of an endothelial cell.

More particularly, the present invention relates to the use of an agent capable of modulating the functional activity of a C-reactive protein in the manufacture of a medicament for the therapeutic and/or prophylactic treatment of a condition, or a predisposition to the development of a condition, characterised by an aberrant inflammatory response in a mammal wherein down-regulating the functional activity of said C-reactive protein down-regulates the adhesion molecule expression of an endothelial cell.

In another preferred embodiment the present invention relates to the use of an agent capable of modulating the functional activity of a C-reactive protein in the manufacture of a medicament for the therapeutic and/or prophylactic treatment of atherosclerosis, atherosclerotic cardiovascular disease, inflammatory cardiovascular disease, diabetic vascular complication or a chronic inflammatory disease in a mammal wherein down-regulating the functional activity of said C-reactive protein down-regulates the adhesion molecule expression of the vascular endothelial cells.

In yet another further aspect, the present invention contemplates a pharmaceutical composition comprising the modulatory agent as hereinbefore defined together with one or more pharmaceutically acceptable carriers and/or diluents.

### BRIEF DESCRIPTION OF THE DRAWINGS

FIG. 1 shows C-reactive protein induced adhesion molecule expression. A. Flow cytometry profiles show cell-surface expression of E-selectin, ICAM-1 and VCAM-1 in HUVEC treated with C-reactive protein (10 μg/ml) for 5 hrs (shaded profiles). The basal levels (solid lines) and negative controls with the isotype-matched, nonrelevant antibodies (dotted lines) are also shown. The mean fluorescence intensity of the positive cells (region Ml) is shown in (B) and (C). B. Time course of C-reactive protein induced adhesion molecule expression. C. Dose response of C-reactive protein for E-selectin expression in HUVEC stimulated for 5 hrs. *P<0.05.

FIG. 2 shows C-reactive protein-induced adhesion molecule expression is dependent on HUVEC conditioned medium but independent of LPS contamination. A. Flow cytometry profiles show cell-surface expression of E-selectin in HUVEC that were untreated (Nil) or treated for 5 hrs with C-reactive protein (10 μg/ml) in serum free HUVEC-conditioned Opti-MEM medium (−FCS) or M199 medium containing 20% FCS (+FCS). B. Conditioned medium was replaced by fresh medium containing the indicated concentrations of conditioned medium immediately prior to C-reactive protein stimulation. E-selectin expression by HUVEC was then assayed. C. E-selectin was measured in HUVEC stimulated for 5 hrs with the indicated concentrations of untreated C-reactive protein or LPS, or with C-reactive protein or LPS that had been heated at 65° C. for the indicated periods of time in serum free medium. *P<0.05; **P<0.01.

FIG. 3 shows HDL inhibits C-reactive protein-induced adhesion molecule expression. HUVEC were stimulated with C-reactive protein (10 μg/ml) for 5 hrs after preincubation for 16 hrs with varying concentrations of native HDL (A) or rHDL (C), then the expression of adhesion molecules was assayed as indicated. *P<0.05 **P<0.01. B. The mRNA levels of E-selectin, VCAM-1, ICAM-1 and GAPDH (as a control) were assayed by RT-PCR in HUVEC preincubated for 16 hrs in serum free medium with native HDL (1 mg/ml) or rHDL (10 μg /ml) followed by C-reactive protein stimulation for the indicated time.

FIG. 4 shows inhibitory activity of HDL on C-reactive protein is reproduced by PLPC alone. A. HUVEC were incubated for 16 hrs in serum free medium with the indicated concentrations of native HDL, rHDL, PLPC or Apo-A1 and stimulated for a further 5 hrs with C-reactive protein (10 μg/ml), then the expression of adhesion molecules was assayed as indicated. B. Representative histogram of C-reactive protein-induced E-selectin expression (stimulated as in A) following 16 hrs preincubation in the presence or absence of PLPC (35 μM).

FIG. 5 shows HDL inhibits adherence of U937 cells to BAEC. BAECs were treated with TNF (0.5 ng/ml) or C-reactive protein (10 μg/ml) for 24 hrs in the presence or absence of rHDL (10 μg/ml). The pre-labelled U937 cells were incubated with the treated-BAECs for 30 min. A. Adherence of U937 cells was microscopically photographed (20×), and (B.) determined by visually counting 4 microscopic fields per well in triplicate. *P<0.001.

FIG. 6 shows PLPC activity is dependent on HUVEC conditioned medium. A. Unconditioned rHDL or PLPC SUVs were preincubated with HUVEC for 16 hrs or 1 hr with or without washout (W/O), or the HUVEC-conditioned (HC) rHDL or PLPC SUVs (detailed in ‘Methods’) were preincubated with HUVEC for 1 hr, the cells were then stimulated with C-reactive protein for 5 hrs and E-selectin expression was assayed. *P<0.05. B. Mass spectrometric analysis of PLPC prior to incubation with HUVEC (main panel). The inner panel shows the analysis of the lipid extracted from the serum-free medium following overnight incubation of PLPC with HUVEC.

FIG. 7 shows oxidation of PLPC eliminates the C-reactive protein induced response. The expression of E-selectin was assayed in (A) HUVEC preincubated for 16 hrs with 35 μM PLPC in the presence of varying concentrations of a-tocopherol, or (B) HUVEC stimulated for 5 hrs with either C-reactive protein (10 μg/ml) or TNF (0.1 ng/ml) added simultaneously with 35 μM non-oxidized (non-ox) or oxidized PLPC (ox). *P<0.01.

### DETAILED DESCRIPTION OF THE INVENTION

The present invention is predicated, in part, on the determination that the C-reactive protein-induced expression of the inflammatory adhesion molecules E-selectin, VCAM-1 and ICAM-1 in human umbilical vein endothelial cells (HUVEC) is completely suppressed when the cells are preincubated with high density lipoproteins (HDL). Furthermore, it has been found both that the oxidized form of the phospholipid component of rHDL is the central molecule responsible for the inhibitory effect of HDL on C-reactive protein and that this mechanism is independent of and distinct from the mechanism which regulates cytokine mediated adhesion molecule expression. The elucidation of this cellular signalling mechanism now facilitates the rational design of methodology directed to treating adverse or unwanted vascular inflammatory responses.

Accordingly, one aspect of the present invention provides a method of modulation of the endothelial cell proinflammatory phenotype which method comprises administration of an effective amount of an agent for a time and under conditions sufficient to modulate the functional activity of a C-reactive protein wherein down-regulating the functional activity of said C-reactive protein down-regulates said inflammatory phenotype.

Reference to an “inflammatory phenotype” should be understood to mean any one or more of the cellular characteristics which are associated with inflammation. Examples of such characteristics include, but are not limited to, increasing the secretion of proinflammatory soluble factors (eg. monocyte chemoattractant protein), reducing nitric oxide bioactivity, inducing adhesion molecule expression (eg. intercellular adhesion molecule—ICAM-1; vascular adhesion molecule-1—VCAM-1; E-selection). Preferably, said inflammatory phenotype is upregulation of adhesion molecule expression.

The present invention therefore more particularly provides a method of modulation of endothelial cell adhesion molecule expression, which method comprises administration of an effective amount of an agent for a time and under conditions sufficient to modulate the functional activity of a C-reactive protein wherein down-regulating the functional activity of said C-reactive protein down-regulates said adhesion molecule expression.

Reference to “adhesion molecule” should be understood as a reference to a molecule which mediates the binding of a cell to another cell or to a protein such as an extracellular matrix protein. Examples of adhesion molecules include, but are not limited to, integrins, selectins (eg. E-selectin, P-selectin), members of the immunoglobulin-gene superfamily (eg. VCAM-1, ICAM-1) and CD44. Preferably, said adhesion molecule is ICAM-1, VCAM-1 and/or E-selectin.

Reference to “endothelial cell” should be understood as a reference to the endothelial cells which line the blood vessels, lymphatics or other serous cavities such as fluid filled cavities. The phrase “endothelial cell” should also be understood as a reference to endothelial cell mutants. “Mutants” include, but are not limited to, endothelial cells which have been naturally or non-naturally modified such as cells which are genetically modified.

It should also be understood that the endothelial cells of the present invention may be at any differentiative stage of development. Accordingly, although committed to differentiating along the endothelial cell lineage, the cells may be immature and therefore partially functional in the absence of further differentiation. Preferably, the subject of endothelial cell is a vascular endothelial cell.

The present invention therefore more preferably provides a method of modulation of vascular endothelial cell adhesion molecule expression, which method comprises administration of an effective amount of an agent for a time and under conditions sufficient to modulate the functional activity of a C-reactive protein wherein down-regulating the functional activity of said C-reactive protein down-regulates said adhesion molecule expression.

Reference to “C-reactive protein” should be understood as a reference to all forms of this protein and to functional derivatives or homologues thereof. This includes, for example, any isoforms which arise from alternative splicing of the subject C-reactive protein mRNA or mutants or polymorphic variants of these proteins. Without limiting the present invention to any one theory or mode of action, C-reactive protein is an acidic, pentameric, heat sensitive protein of approximately 118 kDa. It is one of the acute phase proteins.

Reference to “modulating” the endothelial cell proinflammatory phenotype should be understood as a reference to up-regulating or down-regulating this endothelial cell phenotype. Reference to “down-regulating” the endothelial cell proinflammatory phenotype should therefore be understood as a reference to preventing, reducing (eg. slowing) or otherwise inhibiting (e.g. delaying or terminating) the subject phenotype (for example retarding or preventing endothelial cell adhesion molecule expression) while reference to “up-regulating” should be understood to have the converse meaning and includes prolonging or enhancing the subject response. Although the preferred method is to down-regulate the inflammatory phenotype, in order to down-regulate an unwanted inflammatory response, the present invention nevertheless extends to up-regulating the inflammatory phenotype in order to up-regulate an inflammatory response in circumstances where it is desired that an inflammatory response occur. For example, one may seek to upregulate an inflammatory response in order to treat infections (e.g. protect against acute infections), to facilitate or enhance cancer therapy, to increase vascular regeneration during wound healing or to rescue myocardial infarction.

It should be understood that the onset of an endothelial cell proinflammatory phenotype, in particular endothelial cell adhesion molecule expression, may correlate to an entirely aberrant response or it may be one which in fact correlates to a normal physiological response but is nevertheless unwanted. Accordingly, the present invention provides a means of down-regulating an unwanted endothelial cell inflammatory response, in particular a vascular endothelial cell inflammatory response, irrespective of whether it correlates to a physiologically normal response versus an entirely aberrant and destructive response.

In a preferred embodiment, the present invention provides a method of down-regulating vascular endothelial cell adhesion molecule expression, which method comprises administration of an effective amount of an agent for a time and under conditions sufficient to down-regulate the functional activity of C-reactive protein.

Reference to modulating the “functioning” of C-reactive protein should be understood as a reference to modulating the level of C-reactive protein activity as opposed to the concentration of C-reactive protein per se. Although a decrease in the concentration of C-reactive protein will generally correlate to a decrease in the level of C-reactive protein functional activity which is observed, the person skilled in the art would also understand that decreases in the level of activity can be achieved by means other than merely decreasing absolute C-reactive protein concentrations. For example, one might utilise means of decreasing the half life of C-reactive protein or sterically hindering the binding of this molecule to it substrate.

It should also be understood that reference to modulation of C-reactive protein activity, in particular its down-regulation, does not necessarily mean that the activity of this molecule needs to be returned to physiologically normal levels. Rather, the level need only be one which is changed relative to the pretreatment level. Accordingly, the method of the present invention may be applied to partially reduce unwanted vascular endothelial cell proinflammatory activity in some situations while in other situations it may be desirable or necessary to completely eliminate vascular endothelial cell proinflammatory activity. The subject modulation may be transient or long term, depending on the requirements of the particular situation.

It should be understood that reference to “effective amount” means the amount necessary to at least partly attain the desired response. The amount may vary depending on the health and physical condition of the cellular population and/or individual being treated, the taxonomic group of the cellular population and/or individual being treated, the degree of up or down-regulation which is desired, the formulation of the composition which is utilised, the assessment of the medical situation and other relevant factors. Accordingly, it is expected that this level may vary between individual situations, thereby falling in a broad range, which can be determined through routine trials.

Modulating C-reactive protein activity may be achieved by any suitable means including, but not limited to:
(i) Modulating absolute levels of C-reactive protein such that either more or less C-reactive protein is present in the cellular environment.
(ii) Agonising or antagonising C-reactive protein functional activity such that the functional effectiveness (ie. the overall activity) of C-reactive protein is either increased or decreased. For example, increasing the half life of C-reactive protein may achieve an increase in the functionally effective level of C-reactive protein without actually necessitating an increase in the absolute concentration of C-reactive protein. Similarly, the partial antagonism of C-reactive protein, for example by dissociating the pentameric form into free subunits or by coupling this molecule to components that introduce some steric hindrance in relation to binding to its target, may act to reduce, although not necessarily eliminate, the functional effectiveness of said C-reactive protein. Accordingly, this may provide a means of down-regulating C-reactive protein functioning without necessarily down-regulating absolute concentrations of C-reactive protein.

In terms of achieving the up or down-regulation of C-reactive protein activity, means for achieving this objective would be well known to the person of skill in the art and include, but are not limited to:
(i) Introducing into a cell a nucleic acid molecule encoding C-reactive protein or in order to up-regulate the capacity of said cell to express C-reactive protein.
(ii) Introducing into a cell a proteinaceous or non-proteinaceous molecule which modulates transcriptional and/or translational regulation of a gene, wherein this gene may be the C-reactive protein gene or functional portion thereof or some other gene or gene region (eg. promoter region) which directly or indirectly modulates the expression of the C-reactive protein gene.
(iii) Introducing into a mammal the C-reactive protein expression product (this should be understood to include the use of C-reactive protein functional derivatives or homologues).
(iv) Introducing a proteinaceous or non-proteinaceous molecule which functions as an antagonist to the C-reactive protein expression product such as native or reconstituted lipoprotein (e.g; HDL or LDL) lipid (in particular phospholipid) steroid, fatty acid (including saturated and unsaturated forms), functional derivatives or oxidised forms of these molecules.
(v) Introducing a proteinaceous or non-proteinaceous molecule which functions as an agonist of the C-reactive protein expression product.

The proteinaceous molecules described above may be derived from any suitable source such as natural, recombinant or synthetic sources and include fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the C-reactive protein expression product or small molecules capable of acting as agonists or antagonists. Chemical agonists may not necessarily be derived from the C-reactive protein expression product but may share certain conformational similarities. Alternatively, chemical agonists may be specifically designed to meet certain physiochemical properties. Antagonists may be any compound capable of blocking, inhibiting or otherwise preventing C-reactive protein from carrying out its normal biological function. Antagonists include monoclonal antibodies and antisense nucleic acids which prevent transcription or translation of C-reactive protein genes or mRNA in mammalian cells. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) of C-reactive protein may be created or identified by their ability to suppress the expression of C-reactive protein. The production of antisense oligonucleotides for a given protein is described in, for example, Stein and Cohen, 1988 (Cancer Res 48:2659-68) and van der Krol et al., 1988 (Biotechniques 6:958-976) .

The proteinaceous and non-proteinaceous molecules referred to in points (i)-(v), above, are herein collectively referred to as “modulatory agents”. Preferably, said modulatory agent is native or reconstituted lipoprotein (e.g. HDL or LDL) lipid (in particular phospholipid) steroid, fatty acid (including saturated and unsaturated forms), functional derivatives or oxidised forms of any of these molecules or functional derivative thereof, to the extent that it is sought to decrease C-reactive protein activity.

Without limiting the present invention in any way, HDL (high density lipoprotein) corresponds to one of the classes of lipoprotein found in the blood plasma of may animals. These molecules are also known as a-lipoproteins and exhibit the highest electrophoretic mobility of the lipoproteins. The approximate composition of HDL (% by weight) is 6% unesterified cholesterol, 13% esterified cholesterol, 28% phospholipid, 3% triacylgycerol, 50% protein. Their apolipoprotein composition (% by weight apolipoprotein) is approximately A-I (67%); A-II (22%); C-I and C-II and C-III (5-11 %); E-II and E-III and E-IV (1-2%); trace amounts of D. Discoidal reconstituted HDL comprises apoA-I and PLPC although it should be understood that the present invention extends to the use of any other form of reconstituted HDL. Still without limiting the present invention in any way, by reconstituting HDL for use in the method of the invention, there is minimised the probability of unwanted side-effects which may be linked to the presence of contaminants which may be co-isolated with native HDL. Still further, by using reconstituted HDL there is also minimised the possibility of side-effects related to the heterogeneity of native HDL particles. Nevertheless, native HDL also remains a highly useful C-reactive protein antagonist for use in the present invention. It has also been determined that PLPC, alone, is functional in antagonising C-reactive protein, as are unsaturated phospholipids.

Still without limiting the present invention to any one theory or mode of action, although oxidized phospholipids within oxidized-low density lipoproteins, are generally considered as proinflammatory agonists, recent reports have shown that some oxidized phospholipids inhibit LPS-induced upregulation of inflammatory genes (Leitinger et al (1999), Bochkov et al (2002)). Nevertheless, it has been surprisingly determined that oxidized PLPC is a key molecule in mediating the inhibitory effect on HDL on C-reactive protein proinflammatory activity. Oxidation of phospholipids is thought to result in a conformational change that reveals ‘cryptic’ binding sites to C-reactive protein. Thus, the interaction of HDL with endothelial cells may expose C-reactive protein binding sites by oxidation of the phospholipids within the HDL particles, leading to a competitive inhibition of the interaction between C-reactive protein and the endothelial cell.

Accordingly, in a most preferred embodiment the subject native or reconstituted lipoprotein (e.g. HDL or LDL), lipid (for example PLPC in the form of small unilamellar vesicles), fatty acid (including saturated and unsaturated forms), functional derivatives thereof is oxidized. It should be understood that said oxidation may be complete or partial, although complete oxidation is more preferable.

According to this most preferred embodiment there is provided a method of down-regulating vascular endothelial cell adhesion molecule expression, which method comprises administration of an effective amount of an agent selected from:
(v) HDL
(vi) reconstituted HDL
(vii) PLPC
(viii) unsaturated phospholipid
   or a derivative thereof for a time and under conditions sufficient to down-regulate the functional activity of a C-reactive protein.

Preferably, said HDL, reconstituted HDL, PLPC or unsaturated phospholipid is partially or fully oxidised.

Screening for the modulatory agents hereinbefore defined can be achieved by any one of several suitable methods including, but in no way limited to, contacting a cell comprising the C-reactive protein gene or functional homologue or derivative thereof with an agent and screening for the modulation of C-reactive protein production or functional activity, modulation of the expression of a nucleic acid molecule encoding C-reactive protein or modulation of the activity or expression of a downstream C-reactive protein cellular target. Detecting such modulation can be achieved utilising techniques such as Western blotting, electrophoretic mobility shift assays and/or the readout of reporters of C-reactive protein activity such as luciferases, CAT and the like.

It should be understood that the C-reactive protein gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate C-reactive protein activity, at either the nucleic acid or expression product levels, or the gene may require activation—thereby providing a model useful for, inter alia, screening for agents which up-regulate C-reactive protein expression. Further, to the extent that a C-reactive protein nucleic acid molecule is transfected into a cell, that molecule may comprise the entire C-reactive protein gene or it may merely comprise a portion of the gene such as the portion which regulates expression of the C-reactive protein product. For example, the C-reactive protein promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. In another example, the subject of detection could be a downstream C-reactive protein regulatory target, rather than C-reactive protein itself. Yet another example includes C-reactive protein binding sites ligated to a minimal reporter. Modulation of C-reactive protein activity can be detected by screening for the modulation of proinflammatory adhesion molecule expression. This is an example of an indirect system where modulation of C-reactive protein expression, per se, is not the subject of detection. Rather, modulation of the down-stream activity which C-reactive protein regulates is monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the C-reactive protein nucleic acid molecule or expression product itself or which modulate the expression of an upstream molecule, which upstream molecule subsequently modulates C-reactive protein expression or expression product activity. Accordingly, these methods provide a mechanism of detecting agents which either directly or indirectly modulate C-reactive protein expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention said agent is associated with a molecule which permits its targeting to a localised region, for example the cardiovascular region.

The subject proteinaceous or non-proteinaceous molecule may act either directly or indirectly to modulate the expression of C-reactive protein or the activity of the C-reactive protein expression product. Said molecule acts directly if it associates with the C-reactive protein nucleic acid molecule or expression product to modulate expression or activity, respectively. Said molecule acts indirectly if it associates with a molecule other than the C-reactive protein nucleic acid molecule or expression product which other molecule either directly or indirectly modulates the expression or activity of the C-reactive protein nucleic acid molecule or expression product, respectively. Accordingly, the method of the present invention encompasses the regulation of C-reactive protein nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example C-reactive protein, HDL, PLPC or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place.

Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, HDL, or derivative thereof may be fused to a molecule to facilitate its localisation to a particular site. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” of C-reactive protein or a modulatory agent should be understood to mean molecules which exhibit at least some of the functional activity of the form of C-reactive protein of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of C-reactive protein or modulatory agent which exhibits similar and suitable functional characteristics to that of the molecule which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing C-reactive protein analogues which exhibit properties such as more potent pharmacological effects. C-reactive protein or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of C-reactive protein or HDL, for example, one may identify and utilise molecules which function agonistically or antagonistically to C-reactive protein in order to up or down-regulate the functional activity of C-reactive protein in relation to modulating endothelial cell adhesion molecule expression. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of C-reactive protein capable of acting as agonists or antagonists. Chemical agonists may not necessarily be derived from C-reactive protein but may share certain conformational similarities. Alternatively, chemical agonists may be specifically designed to mimic certain physiochemical properties of C-reactive protein.

Antagonists may be any compound capable of blocking, inhibiting or otherwise preventing C-reactive protein from carrying out its normal biological functions. Antagonists include monoclonal antibodies specific for C-reactive protein or parts of C-reactive protein. Preferably, said antagonist is HDL, reconstituted HDL, PLPC, an unsaturated phospholipid or an oxidised form of one of these molecules.

Analogues of C-reactive protein or of C-reactive protein agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional amino acid | Code |
|---|---|
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl-Nmbc | |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stablise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Modulation of said C-reactive protein functional levels may be achieved via the administration of C-reactive protein, a nucleic acid molecule encoding C-reactive protein or an agent which effects modulation of C-reactive protein activity or C-reactive protein gene expression (herein collectively referred to as “modulatory agents”). Preferably, the subject method is utilised to down-regulate the inflammatory response in a mammal.

To this end, the method of the present invention contemplates the modulation of endothelial cell proinflammatory activity both in vitro and in vivo. Although the preferred method is to treat an individual in vivo it should nevertheless be understood that it may be desirable that the method of the invention may be applied in an in vitro environment, for example to provide an in vitro model for the analysis of inflammation related vascular aberrancies such as the formation of vascular lesions. In another example, the application of the method of the present invention to an in vitro environment may extend to providing a readout mechanism for screening technologies such as those hereinbefore described. That is, molecules identified utilising these screening techniques can be assayed to observe the extent and/or nature of their functional effect on proinflammatory endothelial cell functioning.

Accordingly, in a related aspect the present invention is directed to modulating the endothelial cell proinflammatory phenotype in a mammal, said method comprising administering to said mammal an effective amount of an agent for a time and under conditions sufficient to modulate the functional activity of a C-reactive protein wherein down-regulating the functional activity of said C-reactive protein down-regulates said inflammatory phenotype.

More particularly, there is provided a method of modulating endothelial cell adhesion molecule expression in a mammal, said method comprising administering to said mammal an effective amount of an agent for. a time and under conditions sufficient to modulate the functional activity of a C-reactive protein wherein down-regulating the functional activity of said C-reactive protein down-regulates said adhesion molecule expression.

Preferably said endothelial cells are vascular endothelial cell. More preferably, said adhesion molecules are E-selectin, VCAM-1 or ICAM-1.

Most preferably, there is provided a method of down-regulating vascular endothelial cell adhesion molecule expression, said method comprising administering an effective amount of an agent for a time and under conditions sufficient to down-regulate the functional activity of a C-reactive protein.

According to this most preferred embodiment, said agent is preferably selected from:
(i) HDL
(ii) reconstituted HDL
(iii) PLPC
(iv) unsaturated phospholipid
   or a functional derivative thereof.

Still more preferably, said HDL, reconstituted HDL, PLPC or unsaturated phospholipid is oxidized.

A further aspect of the present invention relates to the use of the invention in relation to the treatment and/or prophylaxis of disease conditions or other unwanted conditions. Without limiting the present invention to any one theory or mode of action, regulation of the proinflammatory phenotype of endothelial cells, in particular vascular endothelial cells, is a crucial component of the treatment of any inflammatory response.

Accordingly, the present invention is directed to modulating an inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of endothelial cells.

More particularly, there is provided a method of modulating an inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the adhesion molecule expression of endothelial cells.

Preferably said endothelial cells are vascular endothelial cells. More preferably, said adhesion molecules are E-selectin, VCAM-1 or ICAM-1.

Most preferably, there is provided a method of down-regulating an inflammatory response, said method comprising administering an effective amount of an agent for a time and under conditions sufficient to down-regulate the functional activity of a C-reactive protein, wherein down-regulating said C-reactive protein functional activity down-regulates endothelial cell adhesion molecule expression.

According to this most preferred embodiment, said agent is preferably selected from:
(i) HDL
(ii) reconstituted HDL
(iii) PLPC
(iv) unsaturated phospholipid
   or a functional derivative thereof.

Still more preferably, said HDL, reconstituted HDL, PLPC or unsaturated phospholipid is oxidized.

In yet another aspect, the present invention contemplates a method of therapeutically and/or prophylactically treating a condition or a predisposition to the development of a condition, characterised by an aberrant inflammatory response m a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of an endothelial cell.

More particularly, there is provided a method of therapeutically and/or prophylactically treating a condition or a predisposition to the development of a condition, characterised by an aberrant inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the adhesion molecule expression of an endothelial cell.

Preferably said endothelial cells are vascular endothelial cells. More preferably, said adhesion molecules are E-selectin, VCAM-1 or ICAM- 1.

Reference to an “aberrant” inflammatory response should be understood as a reference to an excessive response, an inadequate response or to a physiologically normal response which is inappropriate in that it is unwanted. Examples of excessive or unwanted inflammatory responses include those associated with atherosclerosis, inflammatory cardiovascular disease, atherosclerotic cardiovascular disease, in particular atherosclerotic coronary heart disease and stroke, diabetic vascular complications and other chronic inflammatory diseases such as rheumatoid arthritis and chronic colonic disease. Preferably, said condition is atherosclerosis, atherosclerotic cardiovascular disease, inflammatory cardiovascular disease, diabetic vascular complication or a chronic inflammatory disease.

The present invention therefore preferably provides a method of therapeutically and/or prophylactically treating atherosclerosis, atherosclerotic cardiovascular disease, inflammatory cardiovascular disease, diabetic vascular complication or a chronic inflammatory disease in a mammal, said method comprising administering to said mammal an effective amount of an agent for a time and under conditions sufficient to down-regulate the functional activity of a C-reactive protein wherein down-regulating the functional activity of said C-reactive protein down-regulates the adhesion molecule expression of the vascular endothelial cells.

Preferably, said cardiovascular disease is inflammatory coronary heart disease.

In another preferred embodiment, said condition is obesity, diabetes and/or age. Patients exhibiting these conditions correspond to patients exhibiting a predisposition to the development of a condition characterised by an inflammatory response. Without limiting the present invention to any one theory or mode of action, these are examples of conditions which decrease the quantity and/or quality of HDL. Accordingly, they are often associated with an increase in C-reactive protein concentrations which can ultimately disturb the anti- vs pro-inflammatory balance thereby contributing to the development of inflammatory cardiovascular disease, for example. Accordingly, the method of the present invention may be valuable as a prophylactic measure to be applied to patients exhibiting this type of predisposition.

According to these most preferred embodiments, said down-regulatory inducing agents are selected from:
(i) HDL
(ii) reconstituted HDL
(iii) PLPC
(iv) unsaturated phospholipid
   or a functional derivative thereof.

Preferably, said HDL, reconstituted HDL, PLPC or unsaturated phospholipid is partially or fully oxidized.

In yet another preferred embodiment, the present invention contemplates a method of therapeutically and/or prophylactically treating a condition or a predisposition to the development of a condition, which condition is characterised by an inadequate inflammatory response in a mammal, said method comprising administering to said mammal an effective amount of an agent for a time and under conditions sufficient to up-regulate the functional activity of a C-reactive protein wherein up-regulating the functional activity of said C-reactive protein up-regulates the proinflammatory phenotype of an endothelial cell.

Preferably, said condition is an infection, cancer, one which requires increased vascular regeneration (such as wound healing) or myocardial infarction.

Preferably, said endothelial cell is a vascular endothelial cell. More preferably, said proinflammatory phenotype is the up-regulation of adhesion molecule expression. Still more preferably, said adhesion molecules are E-selectin, VCAM-1 or ICAM-1.

The subject of the treatment or prophylaxis is generally a mammal such as but not limited to human, primate, livestock animal (eg. sheep, cow, horse, donkey, pig), companion animal (eg. dog, cat), laboratory test animal (eg. mouse, rabbit, rat, guinea pig, hamster), captive wild animal (eg. fox, deer). Preferably the mammal is a human or primate. Most preferably the mammal is a human.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome. For example, one may combine the method of the present invention with insulin treatment, to the extent that a cardiovascular heart disease patient is a diabetic.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), respiratory, transdermal, intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, transdermally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant. Preferably, said means of administration is inhalation with respect to the treatment of airway inflammation and intravenously, intramuscularly or transdermally for other conditions.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of an agent capable of modulating the functional activity of a C-reactive protein in the manufacture of a medicament for the therapeutic and/or prophylactic treatment of a condition, or a predisposition to the development of a condition, characterised by an aberrant inflammatory response in a mammal wherein down-regulating the functional activity of said C-reactive protein down-regulates the proinflammatory phenotype of an endothelial cell.

More particularly, the present invention relates to the use of an agent capable of modulating the functional activity of a C-reactive protein in the manufacture of a medicament for the therapeutic and/or prophylactic treatment of a condition, or a predisposition to the development of a condition, characterised by an aberrant inflammatory response in a mammal wherein down-regulating the functional activity of said C-reactive protein down-regulates the adhesion molecule expression of an endothelial cell.

Preferably said endothelial cells are vascular endothelial cells. More preferably, said adhesion molecules are E-selectin, VCAM-1 or ICAM- 1.

In another preferred embodiment the present invention relates to the use of an agent capable of modulating the functional activity of a C-reactive protein in the manufacture of a medicament for the therapeutic and/or prophylactic treatment of atherosclerosis, atherosclerotic cardiovascular disease, inflammatory cardiovascular disease, diabetic vascular complication or a chronic inflammatory disease in a mammal wherein down-regulating the functional activity of said C-reactive protein down-regulates the adhesion molecule expression of the vascular endothelial cells.

Preferably, said cardiovascular disease is inflammatory coronary heart disease or stoke.

In yet another preferred embodiment, said condition is obesity, diabetes and/or age. Patients exhibiting these conditions correspond to patients exhibiting a predisposition to the development of a condition characterised by an inflammatory response.

In still another aspect said functional activity is upregulated and said condition is an infection, cancer, one which requires increased vascular regeneration (such as wound healing) or myocardial infarction.

According to these most preferred embodiments, said down-regulatory inducing agents are selected from:
(i) HDL
(ii) reconstituted HDL
(iii) PLPC
(iv) unsaturated phospholipid
   or a functional derivative thereof.

Preferably, said HDL, reconstituted HDL, PLPC or unsaturated phospholipid is partially or fully oxidized.

In yet another further aspect, the present invention contemplates a pharmaceutical composition comprising the modulatory agent as hereinbefore defined together with one or more pharmaceutically acceptable carriers and/or diluents. These agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Various methods of transferring or delivering DNA to cells for expression of the gene product protein, otherwise referred to as gene therapy, are disclosed in Gene Transfer into Mammalian Somatic Cells in vivo, N. Yang, Grit. Rev. Biotech. 12(4):335-356 (1992), which is hereby incorporated by reference.

Strategies for treating these medical problems with gene therapy include therapeutic strategies such as identifying a defective gene or protein and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that for a C-reactive protein antagonist may be placed in a patient and thus prevent or mitigate the occurrence of adverse vascular endothelial cell inflammation.

Many protocols for transfer of genetic regulatory sequences are envisioned in this invention. Transfection of promoter sequences, or other sequences which would modulate the expression and/or activity of C-reactive protein are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate the subject gene.

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer.

Chemical methods of gene therapy may involve a lipid based compound, not necessarily a liposome, to ferry the DNA across the cell membrane. Lipofectins or cytofectins, lipid-based positive ions that bind to negatively charged DNA, may be used to cross the cell membrane and provide the DNA into the interior of the cell. Another chemical method may include receptor-based endocytosis, which involves binding a specific ligand to a cell surface receptor and enveloping and transporting it across the cell membrane.

Many gene therapy methodologies employ viral vectors such as retrovirus vectors to insert genes into cells. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors may be selected from the group including, but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors and are preferred. Adenoviral vectors may be delivered bound to an antibody that is in turn bound to collagen coated stents.

Mechanical methods of DNA delivery may be employed and include, but are not limited to, fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun”, inorganic chemical approaches such as calcium phosphate transfection and plasmid DNA incorporated into polymer coated stents. Ligand-mediated gene therapy, may also be employed involving complexing the DNA with specific ligands to form ligand-DNA conjugates, to direct the DNA to a specific cell or tissue.

The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Gene regulation of C-reactive protein functioning may be accomplished by administering compounds that bind the C-reactive protein gene, for example, or control regions associated with the C-reactive protein gene, or corresponding RNA transcript to modify the rate of transcription or translation. Additionally, cells transfected with a DNA sequence encoding a C-reactive protein antagonist or agonist may be administered to a patient to provide an in vivo source of a C-reactive protein regulator. For example, cells may be transfected with a vector containing a nucleic acid sequence encoding a C-reactive protein regulator.

The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. DNA sequence is operatively linked to an expression control sequence to form an expression vector capable of gene regulation. The transfected cells may be cells derived from the patient's normal tissue, the patient's diseased tissue (such as diseased vascular tissue), or may be non-patient cells. For example, blood vessel cells removed from a patient can be transfected with a vector capable of expressing a regulatory molecule of the present invention, and be re-introduced into the patient. Patients may be human or non-human animals. Cells may also be transfected by non-vector, or physical or chemical methods known in the art such as electroporation, incorporation, or via a “gene gun”. Additionally, DNA may be directly injected, without the aid of a carrier, into a patient.

The gene therapy protocol for transfecting a regulatory molecule into a patient may either be through integration of the regulatory molecule's DNA into the genome of the cells, into minichromosomes or as a separate replicating or non-replicating DNA construct in the cytoplasm or nucleoplasm of the cell. Modulation of gene expression and/or activity may continue for a long period of time or may be reinjected periodically to maintain a desired level of gene expression and/or activity in the cell, the tissue or organ.

The modulated cells may replace existing cells such that the existing biological functioning of the cells is modulated. Alternatively, the modulated cells may be used to infiltrate existing regions of disease to halt progression of the disease. The replaced cells may be tissue specific for the condition to be treated. They may also be stem cells, which can be induced to differentiate along a specific lineage.

Yet another aspect of the present invention relates to the agent as hereinbefore defined, when used in the method of the present invention.

The present invention is further defined by the following non-limiting Example.

### EXAMPLE 1

High density lipoproteins neutralise C-reactive protein proinflammatory activity

Material and Methods

Cell Culture and Flow Cytometry Analysis.

Human umbilical vein endothelial cells (HUVEC) and bovine aortic endothelial cells (BAEC) were isolated and cultured as described previously (Wall et al (1978)). Cells were used at passages 2 to 3 for HUVEC and passages 3-10 for BAEC. For detection of adhesion molecules, HUVEC were incubated overnight in Opti-MEM serum free medium (Gibco, Invitrogen Corporation) in the presence or absence of HDL and then treated with recombinant human C-reactive protein (Calbiochem) for 5 hrs unless indicated otherwise. After the treatment, cells were washed with medium M199 and incubated with primary monoclonal antibodies against E-selectin, VCAM-1, ICAM-1 or isotype-matched nonrelevant control antibodies for 30 min as described previously (Gamble et al (1993)). Cells were then incubated with fluorescein isothiocyanate-conjugated secondary antibody for 30 min. The cells were then harvested by trypsinization and fixed in 2.5% formaldehyde (antibody binding prior to trypsinizing has been reported to prevent the partial hydrolyzation of the surface adhesion molecules (Grabner et al (2000))). The expression of cell-surface adhesion molecules was measured as fluorescence intensity by use of a Coulter Epics Profile XL flow cytometer. Unless stated otherwise, the results represent mean fluorescence of the positive population±SEM from one experiment and are representative at least three independent experiments. Differences between means were evaluated by Student's t-tests. ANOVA was used to identify statistical significance of multiple comparisons.

Adherence of U937 Cell to Endothelial Cells.

U937 cells (CRL 1593.2; ATCC) were colorimetrically labelled with 0.2 mg/ml 3-(4,5-dimethylthiazol-2-yl)-2,5-diphenyltetrazolium bromide (MTT; Promega) in normal culture medium for 30 min at 37° C. The cells were collected by low-speed centrifugation and resuspended at a density of 2×10⁵ cells/ml in medium without FCS. BAECs were seeded into 24-well plates and treated as desired in triplicate. After the treatment, BAECs were washed twice with RPMI-1640 medium. The MTT-labelled U937 cell suspension (200 μl/well) was then added into the BAEC cultures and incubated for 30 min at 37° C. Non-adherent cells were removed by rinsing the plates three times with PBS, and the number of adherent cells was counted under microscopy at least 4 fields per well.

Isolation and Preparation of Lipoproteins and Small Unilamellar Vesicles.

As described previously (Ashby et al (1998)) the lipoproteins were isolated from normal healthy adult donors by sequential ultracentrifugation in their appropriate density range: total HDL 1.07<d<1.21 and LDL 1.019<d<1.055 g/ml. The resulting preparations of lipoproteins were dialyzed against endotoxin-free PBS (pH 7.4) prior to use. Discoidal reconstituted HDL containing apoA-I and PLPC, were prepared by the cholate dialysis method described by Matz (1982). Small unilamellar vesicles (SUVs) containing either PLPC or 1-palmitoyl-2-oleoyl-sn-glycero-3-phosphocholine (POPC), in butylated hydroxytoluene (BHT) (molar ratio of PC to BHT, 10:1), were prepared in PBS precisely as described by Jonas (1986). Oxidation of SUV was produced by incubation with 5 μM CuSO₄ in the absence of BHT for 48 hours at 37° C.

Mass Spectrometry.

Samples of phospholipid were extracted with CHCl₃:CH₃OH (2:1), diluted 10-fold in acetonitrile+0.05% formic acid. Samples (20 μl) were infused at 10 μl/min into a PE/Sciex API-100 electrospray-ionization mass spectrometer (PE Biosystems, Melbourne, Australia) with ionization at 4,000V and the orifice set at 65V and the spectra acquired (range 200-1000 Th at 0.1 Th resolution).

RT-PCR.

The primers used to amplify E-selectin, VCAM-1 and ICAM-1 were as described in Meagher et al (1994) and were designed to span intron-exon boundaries. Total RNA was extracted from HUVEC using TRIzol (Gibco BRL) according to the manufacturers instructions. First-strand cDNA was synthesised from 1 μg total RNA using Omniscript reverse transcriptase (QIAGEN) and ADAPTOR primer (Geneworks). E-selectin, VCAM-1 and ICAM-1 were amplified over 27 cycles with an internal GAPDH control. Amplified products were visualised by electrophoresis on 1.5% agarose gel stained with ethidium bromide.

Results

C-Reactive Protein Stimulates Adhesion Molecule Expression in Conditioned Serum-Free Medium.

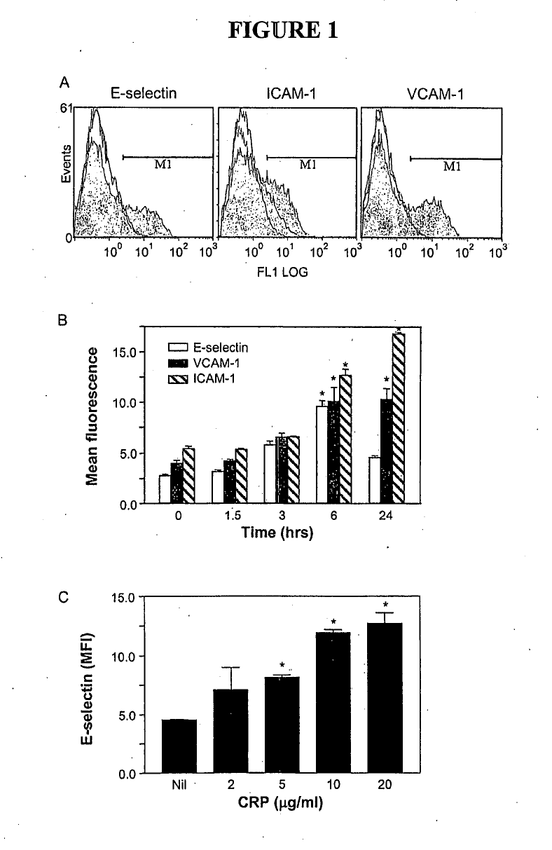

As the induction of adhesion molecules by endothelial cells is critical for proinflammatory reactions in the vasculature, the effect of C-reactive protein on expression of VCAM-1, ICAM-1 and E-selectin in HUVEC was examined. Treatment of HUVEC with C-reactive protein resulted in a significant increase in the cell surface expression of E-selectin VCAM-1 and ICAM-1 (FIG. 1A). The time-course of C-reactive protein induced adhesion protein expression was similar to the effect of cytokines such as TNF (FIG. 1B). The activity of C-reactive protein was dose-dependent, reaching a maximum at 10-20 μg/ml (FIG. 1C) ranged in pathophysiological concentrations that are often seen in chronic inflammatory diseases including atherosclerosis.

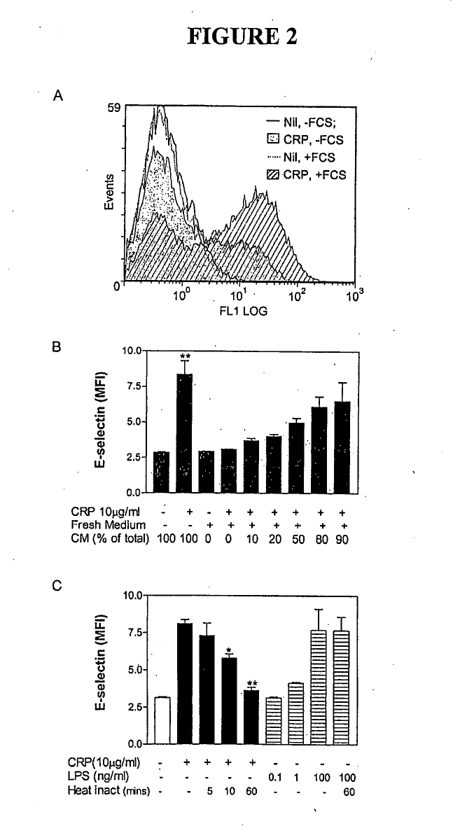

C-reactive protein was capable of inducing adhesion molecule expression in HUVEC cultured in either medium containing 20% FCS or serum-free Opti-MEM medium that had been conditioned by HUVEC for 16 hours (FIG. 2A). However, there was no inflammatory effect when C-reactive protein was added to cells in fresh serum-free medium (FIG. 2B). C-reactive protein induced adhesion molecule expression was restored when HUVEC conditioned medium was added back to the cells in a concentration-dependent manner (FIG. 2B). These data indicate that the effect of C-reactive protein is dependent on a factor that is secreted by endothelial cells or present in serum.

Lipopolysaccharide (LPS) is known to induce adhesion molecule expression, therefore it was necessary to exclude LPS contamination as a factor in the C-reactive protein induced effect. LPS at concentrations of up to 1 ng/ml was insufficient to induce adhesion molecule expression (FIG. 2C), while the contamination of LPS detected in the purified C-reactive protein was below 0.1 ng/ml. Additionally, heating of C-reactive protein at 65° C. significantly decreased the inflammatory effect in a time-dependent manner and heating for 1 hour completely eliminated the C-reactive protein's activity, whereas heating at 65° C. for 1 hour did not alter LPS's effect (FIG. 2C). These data strongly indicate that LPS is not responsible for the observed inflammatory effect of C-reactive protein.

HDL Inhibits C-Reactive Protein-Induced Expression of Adhesion Molecules.

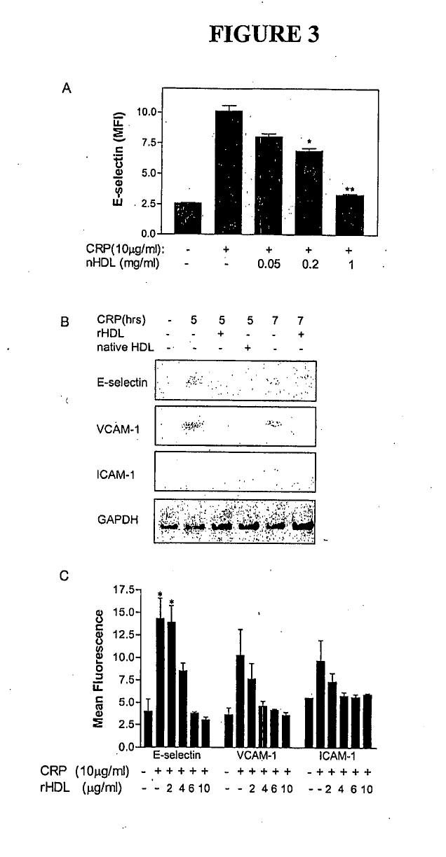

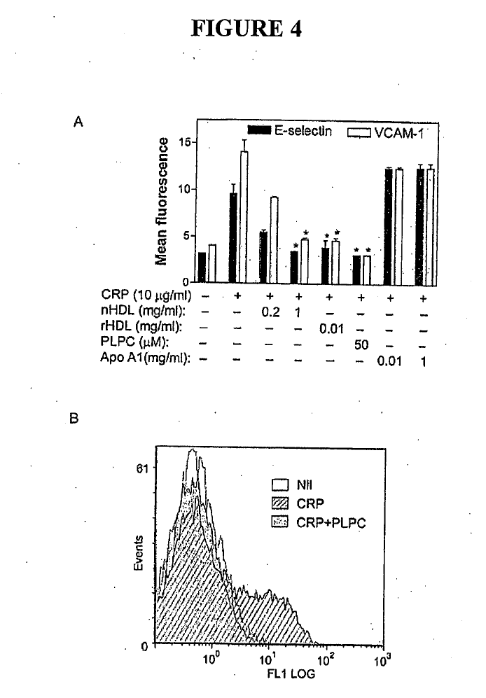

Remarkably, the C-reactive protein-induced expression of adhesion molecules was profoundly inhibited by native HDL in serum-free medium in a concentration-dependent manner (FIG. 3A). Native HDL at a physiological level (1 mg/ml of apoA-I) completely blocked the effect of C-reactive protein on expression of E-selectin, ICAM-1 and VCAM-1. Additionally, the mRNA levels of these adhesion molecules induced by C-reactive protein were also significantly reduced by HDL (FIG. 3B). In order to minimise possible confounding effects of the heterogeneity of native HDL particles (Ashby et al (1998)) and of any co-isolated contaminants, the effects of reconstituted HDL (rHDL) were investigated. Pre-incubation of HUVEC with rHDL containing PLPC and apoA-1 (molar ratio 100:1) resulted in a marked reduction in the C-reactive protein-induced expression of E-selectin, VCAM-1 and ICAM-1 (FIG. 3C). Complete inhibition of the expression by rHDL was attained at a 100-fold lower concentration of HDL particles (10 μg/ml apoA-I) in comparison to native HDL (FIG. 4A). However, treatment with lipid-free apoA-I had no effect (FIG. 4A). In contrast, PLPC presented to the cells as SUVs had a similar inhibitory effect to rHDL (FIG. 4A and 4B), suggesting a major role of the unsaturated phospholipids in the inhibitory activity of HDL. As controls, pre-incubation of HUVEC with LDL or POPC SUVs had no inhibitory effect on C-reactive protein's activity.

HDL Inhibits Adherence of U937 Cells to BAEC.

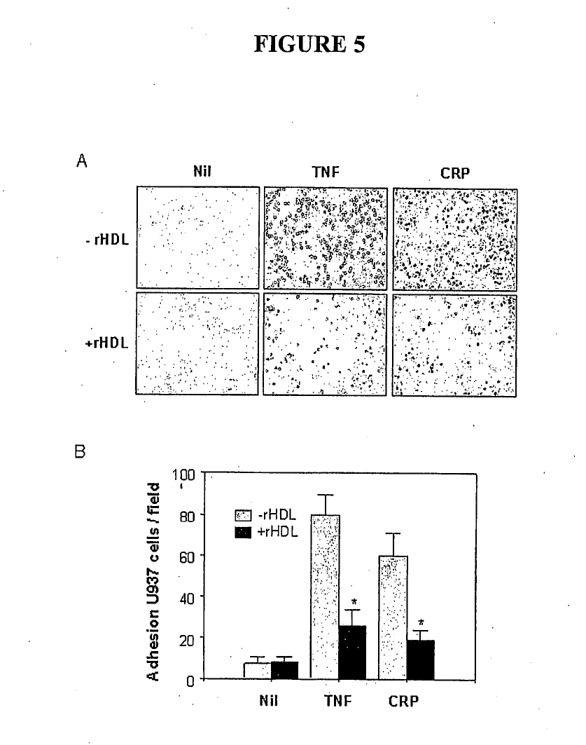

To verify the biological consequences of adhesion molecule expression influenced by C-reactive protein and HDL, leukocyte adherence to aortic endothelial cells was determined. FIG. 5 shows that adhesion of U937 cells increased more than 6-fold following incubation of BAEC with C-reactive protein for 24 hours, comparable to the level of binding following TNF stimulation. The induction of adhesion molecules measured by their mRNA levels was similar to that in HUVEC. Significantly, in the presence of rHDL, the number of U937 cells binding to the C-reactive protein- or TNF-activated BAEC was markedly reduced (FIG. 5).

The Mechanism of HDL Inhibition on C-Reactive Protein Differs from that on Cytokine-Induced Adhesion Molecule Expression.

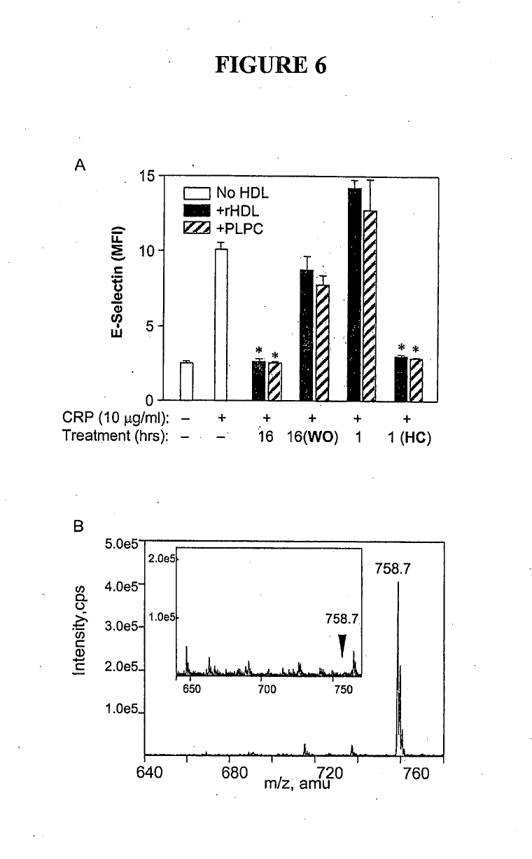

The ability of HDL to inhibit cytokine- (such as TNFα or IL-1) induced adhesion protein expression has been well documented (Baker et al (2000)), thus whether the inhibitory effect of HDL on C-reactive protein is mediated by a common mechanism of inhibition on the adhesion molecule expression induced by cytokines was investigated. In previous reports, it has been shown that a short term pre-incubation (less than 1 hour) with HDL was sufficient for reduction in the TNF-induced expression of VCAM-1 (Baker et al (2000)), and that the inhibition did not require HDL to be physically present during the activation of adhesion molecule expression by TNF (Baker et al (2000), Clay et al(2001)). However, no inhibitory effect on C-reactive protein-induced adhesion molecule expression was discernible following a 1-hour pre-incubation with either rHDL or PLPC (FIG. 6A), or when these reagents were added simultaneously with C-reactive protein. Furthermore, when HDL or PLPC were removed from the medium following a 16-hour pre-incubation before activation of the cells with C-reactive protein, the inhibitory effect did not persist (FIG. 6A). Additionally, in contrast to previous findings of the inability of phospholipids alone to suppress the TNF-induced adhesion protein expression (Cockerill et al (1995), Xia et al (1999)), PLPC had a similar inhibitory effect to whole HDL particles on C-reactive protein (FIG. 4, 6A). Thus, these data suggest different mechanisms underlying HDL inhibition of the C-reactive protein- and cytokine-induced proinflammatory actions.

Oxidation of PLPC is Required to Inhibit the C-Reactive Protein Proinflammatory Effect.

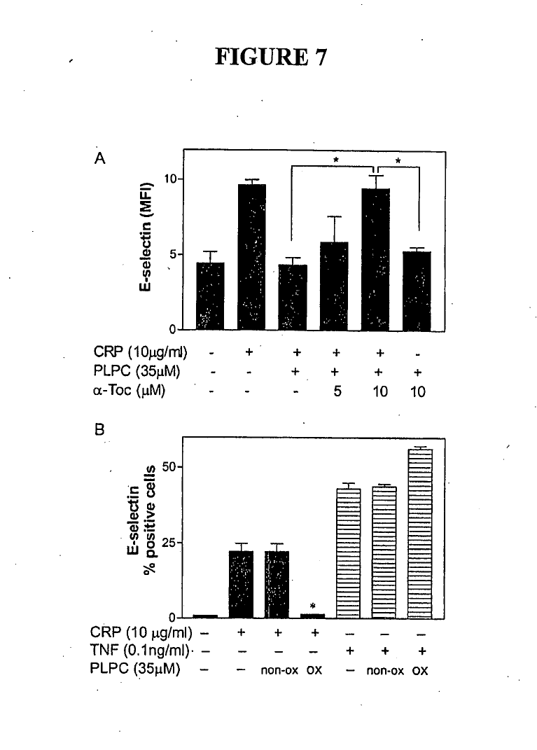

The inhibitory activity of rHDL or PLPC depends on a prolonged pre-incubation or pre-conditioning by cultured HUVEC (FIG. 6A), suggesting that an interaction with endothelial cells is required for the rHDL or PLPC inhibitory effect on C-reactive protein. An investigation was conducted as to whether oxidation of PLPC is involved in the PLPC-dependent inhibition of C-reactive protein proinflammatory effect. Mass spectrometric analysis of unoxidized PLPC revealed a single predominant ion peak at m/z 758.7 (FIG. 6B), which was lost following incubation of PLPC with cultured HUVEC for 16 hrs, presumably due to oxidation (FIG. 6A, inner panel). Interestingly, the PLPC-dependent inhibition of C-reactive protein-induced E-selectin expression was reversed in a dose-dependent manner by the presence of the antioxidants α-tocopherol (FIG. 7A) or nordihydroguaiaretic acid (NDGA). However, PLPC and α-tocopherol alone had no effect on the adhesion molecule expression. These data imply a requirement for oxidized modification in the PLPC-mediated inhibitory effect. To further examine the role of oxidation, PLPC was oxidized in the presence of 5 μM CuSO₄ and a high level of oxidation ascertained by mass spectrometry. When the oxidized PLPC, but not non-oxidized PLPC, was added to cells simultaneously with C-reactive protein, E-selectin expression was abrogated (FIG. 7B). No period of pre-incubation was required for the effect of oxidized PLPC. In contrast, oxidized PLPC did not inhibit TNF activity (FIG. 7B), suggesting a specific effect on C-reactive protein. As a control, POPC that had been exposed to CuSO₄ did not affect C-reactive protein-induced adhesion molecule expression presumably because POPC is less readily oxidized. Taken together, these data strongly indicate that oxidized PLPC is a key molecule accounting for the inhibitory effect of HDL on C-reactive protein proinflammatory activity.

Conclusions

E-selectin, VCAM-1 and ICAM-1 are all induced in HUVEC in the absence of serum following stimulation with C-reactive protein in HUVEC conditioned medium. Thus, the C-reactive protein-induced adhesion molecule expression provides a reliable model for investigation of C-reactive protein proinflammatory activity in vitro. The proinflammatory activity of C-reactive protein can be completely abolished by native HDL at physiological levels. In addition, reconstituted HDL, composed of lipoprotein apoA-I with PLPC as the sole phospholipid, also profoundly inhibited the C-reactive protein-induced expression of E-selectin, VCAM-1 and ICAM-1. Consequently, the physiological significance of these findings was confirmed by the inhibition of C-reactive protein induced adherence of U937 cells to aorticendothelial cells in the presence of HDL (FIG. 5). These findings thus reveal a novel function of HDL to neutralise C-reactive protein-mediated proinflammatory activity on vasculature.

One important finding is that the inhibitory effect of HDL on C-reactive protein differed in several aspects from the effect of HDL on cytokine-induced adhesion molecule expression, suggesting at least two mechanisms of protection against endothelial activation and vascular inflammation by HDL. Interestingly, PLPC alone in the form of SUVs had equivalent inhibitory activity to rHDL, whereas neither lipid-free apoA-I nor POPC had any inhibitory effect. These data differ from previous findings that phospholipids alone were unable to mimic the inhibitory effect of whole HDL particles on the cytokine-induced adhesion molecule expression (Baker et al (2000)), implying a specific role of phospholipids in the protective capacity of HDL against C-reactive protein's proinflammatory action.

The finding that pre-conditioning by incubation with HUVEC was required for the inhibitory effect of PLPC or HDL on. C-reactive protein-induced adhesion molecule expression (FIG. 6A), suggested that pre-conditioning converts the HDL or PLPC from an inactive form to a form that has inhibitory activity. To investigate whether endothelial lipases are involved in this process, tetrahydrolipstatin was used, a specific inhibitor of lipases. It was found that the anti-inflammatory activity of PLPC was not affected by treatment of cells with the lipase inhibitor. Furthermore, the addition of up to 100 μM phosphorylcholine, the product of lipase, to the medium had no effect on the C-reactive protein's activity. Thus, hydrolysis of PLPC is unlikely to account for the anti-inflammatory activity of PLPC or HDL.

The anti-oxidants α-tocopherol and NDGA were able to completely abolish the inhibitory effect of PLPC. Additionally, oxidized PLPC inhibited the proinflammatory effect of C-reactive protein without requiring preincubation with the cells, suggesting an anti-inflammatory potential of this phospholipid. Oxidized phospholipids, especially within oxidised-LDL, are generally considered as proinflammatory agonists. However, the present findings demonstrated that oxidized PLPC was a key molecule in mediating the inhibitory effect of HDL on C-reactive protein proinflammatory activity. Oxidation of phospholipids could result in a conformational change that reveals ‘cryptic’ binding sites to C-reactive protein. Thus, the interaction of HDL with endothelial cells may expose C-reactive protein binding sites by oxidation of the phospholipids within the HDL particles, leading to a competitive inhibition of the interaction between C-reactive protein and endothelial cells.

In summary, a novel function of HDL has been identified that, via oxidation of its principal phospholipid, neutralises the proinflammatory potential of C-reactive protein in endothielial cells, revealing a balance between anti- and proinflammatory actions within the vascular wall.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abbassi O, Kishimoto T. K., McIntire I. V., Anderson D. C., Smith C. W., J. Clin. Invest. 92:2719-2730 (1993)

Ashby D T, Rye K A, Clay M A, et al. Factors influencing the ability of HDL to inhibit expression of vascular cell adhesion molecule-1 in endothelial cells. Arterioscler Thromb Vasc Biol. 18:1450-5 (1998)

Baker P W, Rye K A, Gamble J R, et al. Phospholipid composition of reconstituted high density lipoproteins influences their ability to inhibit endothelial cell adhesion molecule expression. J Lipid Res. 41:1261-7 (2000)

Bochkov V N, Kadl A, Huber J, et al. Protective role of phospholipid oxidation products in endotoxin-induced tissue damage. Nature. 419:77-81 (2002)

Bunin B A, et al. Proc. Natl. Acad. Sci. USA, 91:4708-4712 (1994)

Clay M A, Pyle D H, Rye K A, et al. Time sequence of the inhibition of endothelial adhesion molecule expression by reconstituted high density lipoproteins. Atherosclerosis. 157:23-9 (2001)

Cockerill G W, Rye K A, Gamble J R, et al. High-density lipoproteins inhibit cytokine-induced expression of endothelial cell adhesion molecules. Arterioscler Thromb Vasc Biol. 15:1987-94 (1995)

DeWitt S H, et al. Proc. Natl. Acad. Sci. USA, 90:6909-6913 (1993)

Gamble J R, Khew-Goodall Y, Vadas M A. Transforming growth factor-beta inhibits E-selectin expression on human endothelial cells. J Immunol. 150:4494-503 (1993)

Grabner R, Till U, Heller R. Flow cytometric determination of E-selectin, vascular cell adhesion molecule-1, and intercellular cell adhesion molecule-1 in formaldehyde-fixed endothelial cell monolayers. Cytometry. 40:238-44 (2000)

Johnson-Tidey R. R., McGregor J. L., Taylor P. R., Poston R. N., Am. J. Pathol. 144:952-961 (1994)

Jonas A. Reconstitution of high-density lipoproteins. Methods Enzymol. 128:553-82 (1986)

Leitinger N, Tyner T R, Oslund L, et al. Structurally similar oxidized phospholipids differentially regulate endothelial binding of monocytes and neutrophils. Proc Natl Acad Sci USA. 96:12010-5 (1999)

Matz C E, Jonas A. Micellar complexes of human apolipoprotein A-I with phosphatidylcholines and cholesterol prepared from cholate-lipid dispersions. J Biol Chem. 257:4535-40 (1982)

Meagher L, Mahiouz D, Sugars K, et al. Measurement of mRNA for E-selectin, VCAM-1 and ICAM-1 by reverse transcription and the polymerase chain reaction. J Immunol Methods. 175:237-46 (1994)

O'Brien K. D., Allen M. D., McDonald T. O., Chait A., Harlan J. M., Fishbein D., McCarty J., Furgerson M., Hudkins K., Benjamin C. D., Lobb R., Alpers C. R., J. Clin. Invest. 92:945-951 (1993)

Stein and Cohen, Cancer Res 48:2659-68 (1998)

Van der Wal A. C., Das P. K., Tigges A. J., Becker A. R., Am. J. Pathol. 141:161-168 (1992)

Wall R T, Harker L A, Quadracci L J, et al. Factors influencing endothelial cell proliferation in vitro. J Cell Physiol. 96:203-13 (1978)

Wood K. M., Cadogan M. D., Ranshaw A. I., Parums D. V., Histopathology 22:437-444 (1993)

Xia P, Vadas M A, Rye K A, et al. High density lipoproteins (HDL) interrupt the sphingosine kinase signaling pathway. A possible mechanism for protection against atherosclerosis by HDL. J Biol Chem. 274:33143-7 (1999)

Yang, N., Gene Transfer into Mammalian Somatic Cells in vivo, Grit. Rev. Biotech. 12(4):335-356 (1992)

1. A method of modulating an endothelial cell proinflammatory phenotype which method comprises administration of an effective amount of an agent for a time and under conditions sufficient to modulate the functional activity of a C-reactive protein wherein down-regulating the functional activity of said C-reactive protein down-regulates said inflammatory phenotype.

2. A method of modulating an endothelial cell proinflammatory phenotype in a mammal, said method comprising administering to said mammal an effective amount of an agent for a time and under conditions sufficient to modulate the functional activity of a C-reactive protein wherein down-regulating the functional activity of said C-reactive protein down-regulates said inflammatory phenotype.

3. A method of modulating an inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of an endothelial cell.

4. A method of therapeutically and/or prophylactically treating a condition, or a predisposition to the development of a condition, characterised by an aberrant inflammatory response in a mammal, said method comprising administering to said mammal an effective amount of an agent for a time and under conditions sufficient to modulate the functional activity of a C-reactive protein wherein down-regulating the functional activity of said C-reactive protein down-regulates the proinflammatory phenotype of an endothelial cell.

5. The method according to any one of claims 1 to 4, wherein said proinflammatory phenotype is the up-regulation of adhesion molecule expression.

6. The method according to claim 5, wherein said adhesion molecule is ICAM-1, VCAM-1 or E-selectin.

7. The method according to claim 5 or 6, wherein said endothelial cell is a vascular endothelial cell.

8. The method according to claim 7, wherein said modulation of adhesion molecule expression is down-regulation of expression.

9. The method according to claim 7, wherein said modulation of adhesion molecule expression is up-regulation of expression.

10. The method according to claim 4, wherein said condition is an unwanted inflammatory condition, said endothelial cell is a vascular endothelial cell and said proinflammatory phenotype is adhesion molecule expression which is down-regulated.

11. The method according to claim 10, wherein said adhesion molecule is ICAM-1, VCAM-1 or E-selectin.

12. The method according to claim 11, wherein said inflammatory condition is atherosclerosis, inflammatory cardiovascular disease or atherosclerotic cardiovascular disease.

13. The method according to claim 12, wherein said atherosclerotic cardiovascular disease is atherosclerotic coronary heart disease or stroke.

14. The method according to claim 11, wherein said inflammatory condition is diabetic vascular complications or chronic inflammatory disease such as rheumatoid arthritis or chronic colonic disease.

15. The method according to any one of claims 12 to 14, wherein said mammal has diabetes and is predisposed to the development of said inflammatory condition.

16. The method according to any one of claims 12 to 14, wherein said mammal is obese and is predisposed to the development of said inflammatory condition.

17. The method according to claim 4, wherein said condition is an inadequate inflammatory response, said endothelial cell is a vascular endothelial cell and said proinflammatory phenotype is adhesion molecule expression which is up-regulated.

18. The method according to claim 17 wherein said condition is an infection, cancer, myocardial infarction or said condition is one which requires an increase in vascular regeneration, such as wound healing.

19. The method according to claim 17 or 18, wherein said adhesion molecule is ICAM-1, VCAM-1 or E-selectin.

20. The method according to any one of claims 1 to 7, 9 or 17 to 19, wherein said modulation is upregulation of C-reactive protein functional activity and said up-regulation is achieved by introducing into said mammal a nucleic acid molecule encoding C-reactive protein or functional derivative or homologue thereof or the C-reactive protein expression product or functional derivative or homologue thereof.

21. The method according to any one of claims 1 to 18, wherein said modulation is achieved by introducing to said mammal a proteinaceous or non-proteinaceous molecule which modulates transcriptional and/or translational regulation of the C-reactive protein gene.

22. The method according to any one of claims 1 to 7, 9 or 17 to 19, wherein said modulation is up-regulation of C-reactive protein functional activity and said up-regulation is achieved by contacting said endothelial cell with a proteinaceous or non-proteinaceous molecule which functions as an agonist of the C-reactive protein expression product.

23. The method according to any one of claims 1 to 8 or 10 to 16, wherein said modulation is down-regulation of C-reactive protein functional activity and said down-regulation is achieved by introducing to said mammal a proteinaceous or non-proteinaceous molecule which functions as an antagonist to the C-reactive protein expression product.

24. The method according to claim 23, wherein said antagonist is a lipoprotein.

25. The method according to claim 24, wherein said lipoprotein is native HDL or native LDL.

26. The method according to claim 24, wherein said lipoprotein is reconstituted HDL or reconstituted LDL.

27. The method according to claim 26, wherein said reconstituted HDL is discoidal reconstituted HDL comprising ApoA-I and a phospholipid.

28. The method according to claim 27, wherein said phospholipid is 1-palmitoyl-2-linoleoyl-phospatidyl choline (PLPC).

29. The method according to claim 28, wherein said PLPC and ApoA-I are at a molar ratio of 100:1.

30. The method according to claim 23, wherein said antagonist is a lipid.

31. The method according to claim 30, wherein said lipid is a phospholipid.

32. The method according to claim 31, wherein said phospholipid is a component of HDL or PLPC.

33. The method according to claim 31, wherein said phospholipid is an unsaturated phospholipid.

34. The method according to claim 23 wherein said antagonist is a steroid or fatty acid.

35. The method according to claim 34 wherein said fatty acid is either a saturated or unsaturated form.

36. The method according to any one of claims 24 to 35, wherein said phospholipid component is partially oxidized.

37. The method according to any one of claims 24 to 35, wherein said phospholipid component is fully oxidized.

38. The method according to claim 1, wherein said endothelial cell activity is modulated in vivo.

39. The method according to claim 1, wherein said endothelial cell activity is modulated in vitro.

40. Use of an agent capable of modulating the functional activity of a C-reactive protein in the manufacture of a medicament for the therapeutic and/or prophylactic treatment of a condition, or a predisposition to the development of a condition, characterised by an aberrant inflammatory response in a mammal wherein down-regulating the functional activity of said C-reactive protein down-regulates the proinflammatory phenotype of an endothelial cell.

41. Use of an agent capable of modulating the functional activity of a C-reactive protein in the manufacture of a medicament for the modulation of an endothelial cell proinflammatory phenotype wherein down-regulating the functional activity of said C-reactive protein down-regulates the proinflammatory phenotype of said endothelial cell.

42. Use according to any one of claims 40 or 41, wherein said proinflammatory phenotype is the up-regulation of adhesion molecule expression.

43. Use according to claim 42, wherein said adhesion molecule is ICAM-1, VCAM-1 or E-selectin.

44. Use according to claim 42 or 43, wherein said endothelial cell is a vascular endothelial cell.

45. Use according to claim 44, wherein said modulation of adhesion molecule expression is down-regulation of expression.

46. Use according to claim 44, wherein said modulation of adhesion molecule expression is up-regulation of expression.

47. Use according to claim 40, wherein said condition is an unwanted inflammatory condition, said endothelial cell is a vascular endothelial cell and said proinflammatory phenotype is adhesion molecule expression which is down-regulated.

48. Use according to claim 47, wherein said adhesion molecule is ICAM-1, VCAM-1 or E-selectin.

49. Use according to claim 48, wherein said inflammatory condition is atherosclerosis, inflammatory cardiovascular disease or atherosclerotic cardiovascular disease.

50. Use according to claim 48, wherein said atherosclerotic cardiovascular disease is atherosclerotic coronary heart disease or stroke.

51. Use according to claim 48, wherein said inflammatory condition is diabetic vascular complications or chronic inflammatory disease such as rheumatoid arthritis or chronic colonic disease.

52. Use according to any one of claims 49 to 51, wherein said mammal has diabetes and is predisposed to the development of said inflammatory condition.

53. Use according to any one of claims 49 to 51, wherein said mammal is obese and is predisposed to the development of said inflammatory condition.

54. Use according to claim 40, wherein said condition is an inadequate inflammatory response, said endothelial cell is a vascular endothelial cell and said proinflammatory phenotype is adhesion molecule expression which is up-regulated.

55. Use according to claim 54 wherein said condition is an infection, cancer, myocardial infarction or said condition is one which requires an increase in vascular regeneration, such as wound healing.

56. Use according to claim 54 or 55, wherein said adhesion molecule is ICAM-1, VCAM-1 or E-selectin.

57. Use according to any one of claims 40 to 44, 46 or 54 to 56, wherein said modulation is upregulation or C-reactive protein functional activity and said up-regulation is achieved by introducing into said mammal a nucleic acid molecule encoding C-reactive protein or function derivative or homologue thereof or the C-reactive protein expression product or functional derivative or homologue thereof.

58. Use according to any one of claims 40 to 56, wherein said modulation is achieved by introducing to said mammal a proteinaceous or non-proteinaceous molecule which modulates transcriptional and/or translational regulation of the C-reactive protein gene.

59. Use according to any one of claims 40 to 44, 46 or 54 to 46, wherein said modulation is up-regulation of C-reactive protein functional activity and said up-regulation is achieved by contacting said endothelial cell with a proteinaceous or non-proteinaceous molecule which functions as an agonist of the C-reactive protein expression product.

60. Use according to any one of claims 40 to 45 or 47 to 53, wherein said modulation is down-regulation of C-reactive protein functional activity and said down-regulation is achieved by introducing to said mammal a proteinaceous or non-proteinaceous molecule which functions as an antagonist to the C-reactive protein expression product.

61. Use according to claim 60, wherein said antagonist is a lipoprotein.

62. Use according to claim 61, wherein said lipoprotein is a native HDL or native LDL.

63. Use according to claim 61, wherein said lipoprotein is reconstituted HDL or reconstituted LDL.

64. Use according to claim 63, wherein said reconstituted HDL is discoidal reconstituted HDL comprising ApoA-I and a phospholipid.

65. Use according to claim 64, wherein said phospholipid is 1-palmitoyl-2-linoleoyl-phospatidyl choline (PLPC).

66. Use according to claim 65, wherein said PLPC and ApoA-I are at a molar ratio of 100:1.

67. Use according to claim 60, wherein said antagonist is a lipid.

68. Use according to claim 67, wherein said lipid is a phospholipid.

69. Use according to claim 68, wherein said phospholipid is a component of HDL or PLPC.

70. Use according to claim 67, wherein said phospholipid is an unsaturated phospholipid.

71. The method according to claim 60 wherein said antagonist is a steroid or fatty acid.

72. The method according to claim 71 wherein said fatty acid is either a saturated or unsaturated form.

73. Use according to any one of claims 61 to 70, wherein said phospholipid component is partially oxidized.

74. Use according to any one of claims 61 to 70, wherein said phospholipid component is fully oxidized.

75. A pharmaceutical composition comprising the modulatory agent as hereinbefore defined and one or more pharmaceutically acceptable diluents when used in the method of any one of claims 1 to 39 or in accordance with the use of any one of claims 40 to 74.
